(19) United States
(12) Patent Application Publication
(10) Pub. No.: US 2008/0069913 A1
(43) Pub. Date: Mar. 20, 2008

(54) Anti-Snoring Composition

(75) Inventor: Jean-Christophe Anton, Strasbourg (FR)

Correspondence Address: IP GROUP OF DLA PIPER US LLP, ONE LIBERTY PLACE, 1650 MARKET ST, SUITE 4900, PHILADELPHIA, PA, 19103, US

(73) Assignee: PERSEE MEDICA,, Bailly (FR)

(21) Appl. No.: 11/665,961

(22) PCT Filed: Oct. 5, 2005
(86) PCT No.: PCT/FR05/02444

§ 371 Date: Jun. 25, 2007

(30) Foreign Application Priority Data
Oct. 20, 2004 (FR) 0411153

(51) Int. Cl. A61K 36/73; A61K 31/66; A61K 47/36; A61P 11/00

(52) U.S. Cl. 424/765; 514/114; 514/777

(57) ABSTRACT

An anti-snoring composition including at least one lubricating substance and at least one bioadhesive substance which causes the lubricating substance to adhere to the mucocilliary cells located at pharynx level.

### RELATED APPLICATION

This is a §371 of International Application No. PCT/FR2005/002444, with an international filing date of Oct. 5, 2005 (WO 2006/042926 A1, published Apr. 27, 2006), which is based on French Patent Application No. 04/11153, filed Oct. 20, 2004.

### TECHNICAL FIELD

The technology described herein relates to the field of snoring treatment and, in particular, to compositions for combating snoring.

### BACKGROUND

Snoring is a noise caused by the passage of air through airways constricted at the pharynx level. It is a result of the vibration of the soft palate and the walls of the pharynx under the effect of the abnormally turbulent airflow.

Indeed, the inspiratory air passes through a deformable, elastic and more or less constricted space between the nasal cavity and the trachea, which are both rigid and non-deformable. Flow and pressure modifications emerge in this constriction, which lead to a vibration of the soft palate due to its flapping against the walls of the pharynx (Venturi phenomena). For this vibration to occur, the soft palate must come into contact with the rear wall of the pharynx successively and around 30 times per second, be pushed up by an air current from the nose, and then be pushed back against the palate by any force. In deliberate snoring, this force is a voluntary contraction. During passive nocturnal snoring in prone position, this force is gravity, and when applied to a long and voluminous velum, the latter tends to press spontaneously against the pharynx.

Due to defensive reactions caused by this breathing difficulty, a vicious cycle is created comprising increased breathing effort under the effect of the obstruction, which aims to combat the asphyxia this leads to, but which is responsible for the obstruction which is faster and greater the stronger the effort. Since this voluntary control is impossible during sleep, snoring can only aggravate itself from the moment it begins until waking or half waking stops the constriction and its harmful effects, returning tonus to the oropharynx muscles.

In addition to snoring, the intense breathing effort which the snorer must make causes irritation of the velum against the wall of the pharynx. For this reason, snoring leads to the formation of a palatine uvula oedema which essentially becomes permanent (day and night) over time.

Snoring is caused by several factors which are independent from each other, namely the existence of an excessively long velum, sleeping in supine position and a constriction of the rhino-pharynx following relaxation of the tissue and oedema.

The first factor which causes snoring is the existence of an excessively long velum caused by velum hypertrophy: the palatine uvula elongates, the rear tonsil piers thicken and enlarge into two half curtains which extend the velum downwardly. This hypertrophy is linked to being overweight, which infiltrates tissue with fat, but also to age. This phenomenon sets in after the age of forty. However, it is unusual before this age and snoring in such a case is linked to the existence of a naturally over-developed velum.

The second factor which causes snoring is the supine position (the position of the body sleeping on its back). This position causes the relaxed velum to rest against the rear wall of the pharynx. In this anatomic position, the passage of nasal inspiratory air can easily lift the velum, causing it to float and vibrate. Conversely, when the sleeper is in prone position, the velum falls forward in such a way that there is no risk of coming into contact with the rear or even the side wall of the pharynx; unobstructed in this way, the velum leaves the air corridor behind it open and the air passes easily.

The last element which causes snoring is the constriction of the oral cavity. This constriction is caused by obesity, nasal obstruction and retrognathism.

Obesity is particularly frequent among snorers. Even in mild cases, it rapidly causes a constriction of the free space of the pharynx. Indeed, an overweight subject develops stoutness not only on the outside, but also in naturally free inner spaces, specifically the mouth, the neck and the areas surrounding the upper airways. The congestive pharynx of the overweight subject in his/her fifties is quite distinctive: enormous and full, red and swollen. Alcohol at dinner time also favors snoring by congesting the upper aerodigestive mucosa, which is to say in particular that of the nose and pharynx, and by causing relaxation of tissue due to its anti-spasmodic properties. By a similar mechanism, snoring is aggravated by sleeping drugs. Factors other than obesity or alcohol can also combine to bring about this constriction of the pharyngeal space. These include, for example, certain pathological tongue hypertrophies.

Retrognathism is a shortness of the jaw which, to a greater or lesser extent, constricts the pharynx in the anteroposterior direction, and thus favors vibration of the uvula and the soft parts. Indeed, when the lower jaw is short and the chin projects backwards, the anchoring points of the tongue are pushed back causing the tongue to tilt and easily have a detrimental effect on breathing.

As snoring is a physiological phenomenon which is particularly disturbing for those around the snorer and quite extended throughout the population, a number of products have been developed in recent years. However, the products developed and currently available on the market are not entirely satisfactory as regards their results on snoring, nor as regards their side effects.

Most of these products are administered either by spraying or projection onto the pharyngeal mucosa and their main objective is to lubricate this mucosa. Lubrication is then obtained by the presence of edible oils in these compositions. This technique has two major drawbacks.

The first drawback relates to the risk of oil droplets passing into the lungs and causing lipid pneumonia, which represents an undesirable effect with serious health consequences.

The second drawback relates to the duration of the effect of these products. The mixture is indeed sprayed onto the mucosa layer, which has an intrinsic velocity and pushes the product towards the stomach, away from its site of action. The natural phenomena of swallowing and of saliva production strengthen the phenomenon of removing the oils from their site of action. This is the reason why these products are only satisfactory for a few minutes at the beginning of the night, but in no case for the entire duration of the night and in particular at the middle of the night, during deep sleep, and towards the end of the night.

These observations are confirmed by the following experimental results.

A specialty in the form of a liposome-based liquid preparation, having an anti-snoring effect as its main activity was tested. This preparation contains edible oils (olive, sunflower, etc.) encapsulated in soy lecithin-based liposomes. This preparation, administered when going to bed, aims to lubricate the back of the throat to facilitate the passage of air when breathing in a horizontal position. A certain number of in vitro tests were performed to show whether or not the product persisted over time on a biological membrane. The reference substance selected was distilled water, which is considered to be non-adhesive on physical or biological supports.

Three types of tests were performed: flow measurement on a biological membrane, the test of immersion in artificial saliva and the salting-out test. The rate of flow is the time it takes a given volume of liquid to travel 10 centimetres across a plane tilted at 45°. The saliva immersion test aims to highlight the contact time of the product with the biological membrane when it is subjected to the effect of saliva. Finally, the salting-out test aims to highlight the time it takes the product to salt out a tracer when it is subject to permanent “leaching” by the saliva. These three tests are described in full in the scientific literature and are reference tests for measuring the time a solution remains at its site of action in experimental conditions approximating the physiological conditions of the pharyngeal mucosa.

The results are very clear and evident. For all three tests, the flow time of the liposome-based product is comparable to that of distilled water, which is to say that the product does not remain at its site of action. In all three tests, the time the product remains at its site of action is measured at less than one second. These experimental results confirm observations made among snorers. The liposome-encapsulated edible oil-based product does not remain in place at its site of action, and it can not therefore claim any sort of effectiveness at lubricating the walls of the pharynx.

Finally, none of these products deal with the inflammatory components, oedema and tissue relaxation observed in heavy snorers and which are inherent in these subjects.

It would therefore be advantageous to solve the drawbacks of the prior art by providing a combination of active agents and any other kind of agents that will act on the pharyngeal mucosa to reduce, or even eliminate, the noise of snoring in a definite and quantifiable manner for the entire duration of the sleep.

### SUMMARY

I provide an anti-snoring composition including at least one lubricating substance and at least one bioadhesive substance which causes the lubricating substance to adhere to the mucocilliary cells located at pharynx level.

### DETAILED DESCRIPTION

I provide compositions with properties that enable it to adhere to mucocilliary cells to limit the effect of elimination of the active agents of the composition caused by the velocity of the mucosa and, in particular, elimination of the active agents which lubricate the walls of the pharyngeal mucosa.

I also provide compositions for treating the side effects of snoring which, in turn, contribute to an increase in snoring. These include inflammation and pharyngeal tissue oedemas as well as the relaxation of the pharyngeal mucosa.

Thus, the compositions comprise at least one lubricating substance that lubricates the walls of the pharyngeal mucosa and at least one bioadhesive substance which causes the lubricating substance to adhere to the mucocilliary cells located at the pharynx level.

Surprisingly, I found that the components included in the composition have complementary modes of action, thus providing synergy for more effective action. In particular, I discovered the synergy existing between the lubricating substance and the bioadhesive substance allowing more effective bioadhesion.

“Bioadhesive substance” is understood to mean a biological or synthetic substance capable of “sticking” to mucosa.

To properly understand the synergy existing between the lubricating substance and the bioadhesive substance for preventing snoring, the latter relating to the anatomical and mechanical characteristics of the pharynx and the mucosa, the following recalls the physical and physiological characteristics of the mucus.

Mucus forms a viscous superficial layer on the pharyngeal tissue.

The purpose of mucus is to capture particles inhaled through breathing (dust, viruses, bacteria, fungi and the like), to enable their removal towards the stomach and protect the airway mucosa against irritants or dehydration. Furthermore, mucus plays a very important role in local immune system mechanisms. Mucus, which forms the body's first entrance barrier against foreign elements is permanently renewed to remove all inhaled or ingested particles as quickly as possible. The permanent renewal of mucus is therefore a factor in the rapid removal of ingredients administered in the pharynx.

Mucus includes two layers, namely:
a viscous surface layer, which captures particles, made up of 60-70% mucins (various insoluble substances secreted by caliciform cells and submucosa glands) and 30-40% proteins (especially molecules involved in the immune system such as transferrin, lyzozyme, interferon, kallikrein, immunoglobulins and the like) and
a deep fluid layer made of water-soluble elements forming an aqueous layer allowing the cilia to beat, propelling the surface layer towards the pharynx.

One of the properties of mucus is mucocilliary clearance, which is the combined effect of mucus and the cilia of respiratory mucosa for eliminating foreign particles. This effect is caused by the cilia. These cilia are submerged in the deep fluid layer and they comprise on their ends a small structure resembling a hook, which implants itself in the viscous surface layer of the mucus during movement. The cilia beat in a synchronised manner, their movement being faster in the direction of the pharynx and slower in the opposite direction. The cilia beat by about 1,000 beats/min. and sweep the mucus towards the pharynx at a speed of about 5-20 mm/min.

Thus, the active agents of a traditional product sprayed onto the mucus travel on average 1 cm per minute. It therefore takes 15 minutes, on average, for the layer of mucus containing the active agents to leave its site of action in the pharynx and thus lose its effectiveness.

Through the presence of a substance which favors bioadhesion, not to the mucus but to the mucocilliary cells of the lubricating substance, which favors sliding the velum over the walls of the pharynx, the effectiveness of the anti-snoring composition can be ensured for a longer period, unlike conventional products.

To favor the adhesion of the active agents, and in particular of the lubricating agent, the bioadhesive substance is at least one chosen from the group comprising: polysaccharides, cellulose derivatives, acrylic derivatives or protein derivatives, or any other agent currently known to be a bioadhesive agent.

To increase the bioadhesive property, the bioadhesive substance is advantageously a polysaccharide (natural polymer) belonging to the carrageenan family.

Carrageenans are polysaccharides which form the cell walls of various red algae (rhodophycae) belonging to the Gigartinaceae, Hypneaceae, Furcellariaceae and Polyideaceae families. They comprise long galactan chains, anionic polyelectrolytes. They can have a molecular mass in excess of 106 Daltons.

These linear polymers, formed by disaccharide patterns [AB], include two D-galactose units bonded alternately by (1→3) and (1→4) bonds. These are highly sulphated polysaccharides (20-50%) and the galactose residue can be in the form of 3.6-anhydro.

The sulphate function of carrageenans is a particularly reactive function which produces complex compounds. The carrageenans react to proteins, among others, conferring bioadhesive properties on the proteins. Thus, the carrageenans combine with the proteins contained in the pharyngeal mucosa.

More specifically, the bioadhesive property is generated by an interaction between the polysaccharides and the mucus covering the pharyngeal mucosa. Indeed, due to their chemical nature (polymers with high molecular weight), polysaccharides are hygroscopic. When in contact with mucus, which is very hydrated and has a certain viscosity due to the presence of mucin, polysaccharides quickly swell, creating hydrogen bonds between the hydrophilic groups of the polymer and those of the mucin. This bioadhesive property is then reinforced by the formation of addition bonds due to the sulphate function of the carrageenans. Indeed, the carrageenan sulphate groups, which are particularly reactive, give rise to complexing reactions between the sulphate groups of the polysaccharide and the nitrogen atoms of the mucin, thus forming additional bonds to the hydrogen bonds, known as complexing bonds.

Lambda- and iota-carrageenans are preferably used. The carrageenans are therefore advantageous in that they enable the active agents contained within the anti-snoring composition, in particular, the lubricating substance to remain at their site of action for a long period (with a duration greater than or equal to 8 hours).

As regards lubricating the walls of the pharyngeal mucosa, this is ideally provided by means of a surface-active agent, or of any other currently known lubricating agent or combination of agents.

Surface-active agents are natural or synthetic molecules which have, on the one hand, a lipophilic chain (or hydrophobic part) and, on the other hand, a hydrophilic group (polar part). These components are known as amphiphiles.

The polar part of the surface-active agent is advantageously at least one chosen from the group comprising glucose, sucrose, lactose, glycerol, xylose, peptides, amino acids, nucleotides, the hydrophobic part being at least one chosen from the group comprising fatty acids, fatty alcohol, fatty amine, esters, glycerides, phospholipids.

The hydrophobic part may include phosphatidylcholine, which belongs to the phospholipid family, major components of the membranes surrounding cells. Since it contributes to maintaining the integrity of these membranes and to “repairing” them it plays an essential role in the proper functioning of the body. Moreover, phosphatidylcholine is a major component of bile and plays an important role in the metabolism of fats.

The composition may also comprise one or more anti-inflammatory and/or tonic agent(s) of the mucosa and tissue.

Addition of these active agents to the bioadhesive substance is advantageously encouraged by the emulsifying properties of the selected lubricating substance, in particular of phosphatidylcholine.

To reduce the inflammation and oedema of the pharyngeal tissue, the selected anti-inflammatory agent is advantageously a Filipendula ulmaria extract, and preferably an extract of Filipendula ulmaria flowering tops which contain a group of components including flavonoids and other phenolic heterosides.

To increase the tonicity of the pharyngeal mucosa and tissue, extracts of tannin-rich plants are preferably used to confer astringency properties on the pharyngeal mucosa and tissue. Among these plants, the tonic chosen is advantageously rosehip. Rosehip is an elongated fruit of a small tree called Rosa canina (wild rose).

It is evidently understood that the extracts chosen as anti-inflammatory and tonic agents, and mentioned above, are given as an example. Indeed, any other agent for reducing inflammation of the oedema and/or increasing the tonicity of the pharyngeal mucosa and tissue can be used in the compositions.

Thus, the compositions comprise active agents chosen for their properties for lubricating the pharyngeal mucosa over a long period, associated with a reinforcement of the tonicity of the mucosa and an anti-inflammatory effect.

It is understood that the selected substances are hypoallergenic substances.

Moreover, the compositions advantageously comprise an excipient or have a general formulation, adapted to the intended form of administration. The compositions preferably come in the form of an orally administered foam. Foam can be obtained by dispersing a gas in a small amount of liquid causing the appearance of bubbles, the size of which can vary from about 50 μm to several millimeters.

Indeed, compared with traditional compositions administered in liquid form, foam constitutes a specific galenic form adapted for optimal impregnation of the mucosa.

Indeed, foam has superior flow properties on mucosa to the liquid form which has a tendency, after being sprayed into the mouth, to drain to the bottom of the throat.

Due to its structure, foam significantly increases the surface contact between two incompatible surfaces. Indeed, one of the difficulties found when designing a liquid formulation to be dispersed on a biological membrane is often the incompatibility of the two surfaces. In the specific case of pharyngeal tissue, these are covered in a layer of mucin having hydrophobic properties. Mucin is a protein which performs the function of structural protection, mainly in the airways and digestive tract. Yet, to interact with the mucin and the epithelial cells, the carrageenans must be placed in an aqueous solution which is physically incompatible with the mucin layer. Foam therefore has the advantage of enabling contact between the carrageenans of the solution and the epithelial cells, thus reinforcing bioadhesion. Tests therefore show, in several experiments, that the foam form has greater bioadhesive capability than the liquid form administered in the same quantities. Furthermore, tests have shown that the foam form “lines” the mucosa of the throat and pharynx better. These same tests also show that the contact time between the foam and the mucosa is greater than that of a liquid form.

Another advantage is that the foam holds its structure for longer thanks to, for example, certain molecules contained in plant extracts associated with the product formulation. Certain molecule types contained in the plant extracts used in the formulation (Filipendula Ulmaria and Rosa canina extracts) would promote the stability over time of the foam form due to phenomena of physicochemical interaction. Thus, a foam form would be more stable over time thanks to the effect of flavonoid components. This stability over time reinforces the property listed above by allowing longer contact time between the solution and the epithelial cells of the pharyngeal mucosa.

Finally, the foam form has another advantage which is that is does not diffuse in drops into the airways, as is the case with the traditional forms of liquid spray. These drops are indeed responsible for damage to the lungs and bronchial tubes. Thus, with oily preparations administered in spray form, the appearance of lipid pneumonia with very serious consequences for the health of individuals has been recorded. Lipid pneumonia is a diffused, paused symptommatic pneumopathy, with radio-nodular appearance, predominant at the bases or localized, pseudo-tumoral pneumopathy or oily granuloma. The prognosis is serious and the course is irreversible. By using a foam spray, risks of the solution passing into the airways are avoided.

Furthermore, a large number of patients consider the diffused foam to be more acceptable and easier to use than the liquid form. In the field of snoring, where daily treatment is required, patient acceptance of this type of treatment is a very important factor in compliance and thus in clinical success.

Foam therefore constitutes a preferred galenic form of the anti-snoring compositions. However, it should be understood that the anti-snoring compositions are not limited in any way to this particular galenic form.

The compositions can, in particular, be provided in solid form such as in the form of tablets, lozenges, chewing gum or dissolving paste, or any other instantly/immediately dissolving solid form.

Although less advantageous, as explained above, the compositions can also be provided in liquid form, for example in the form of gel, paste, toothpaste, oral solution and the like.

The percentage by weight of each of the active agents present in the anti-snoring compositions is as follows:
bioadhesive substance: about 0.5 to about 20%
lubricating substance: about 0.5 to about 20%
anti inflammatory agent: about 0.5 to about 8%
tonic agent: about 0.5 to about 8%.

It should be noted that, according to the form in which the compositions are administered, the percentage by weight of the bioadhesive substance will differ. Indeed, in the case of the compositions being administered in foam form, they comprise about 1 to about 5% of bioadhesive substance. In the case of the compositions being administered in solid form, they comprise about 5 to about 20% of bioadhesive substance.

Below is an example of the formulation of a composition in foam form:
Carrageenans about 1 to about 5% preferably about 3%
Phosphatidylcholine about 1 to about 10% preferably about 5%
Meadowsweet (extracts) about 1 to about 5% preferably about 3%
Rosehip about 1 to about 5% preferably about 2%
Excipients: sweeteners, flavorings, preservatives, water.

The composition (bioadhesive preparation) was examined under experimental conditions to verify its bioadhesive properties and compare them to a non-bioadhesive reference substance (distilled water) and a liposome-based commercial preparation (liposome preparation). The measurements carried out included flow time on a biological membrane and immersion in artificial saliva.

The rate of flow is the time it takes for a given volume of liquid to travel 10 cm over a plane tilted at 45°. This test was performed on 3 levels:
on a 20 cm² stainless-steel plate, non-biological membrane;
on a 20 cm² stainless-steel plate covered with a cellulose membrane, biological membrane;
on a 20 cm² stainless-steel plate covered in a cellulose membrane impregnated with a 5% mucin solution.

For this study, 0.5 ml was deposited 0.5 cm from the top edge of the plate, the deposit being made in horizontal position. The chronometer starting line is located 5 cm from the top edge of the plate when the liquid crosses it after tilting the plate to 45°. In the same way, the chronometer stopping line is located 5 cm from the bottom edge of the plate when the liquid crosses it. The measured times are as follows:

| | Stainless-steel | Stainless-steel + | Stainless-steel + |
|---|---|---|---|
| | support | membrane | membrane + mucin |
| Distilled water | <1″ | <1″ | <1″ |
| Liposome | <1″ | <1″ | <1″ |
| preparation | | | |
| Bioadhesive | 1′20 +/− 3″ | 3.27 +/− 10″ | 7.08 +/− 27″ |
| Preparation | | | |

This table shows that the liposome preparation behaves like distilled water, whether on the stainless steel, on the pure biological membrane or with added mucin. Conversely, the bioadhesive preparation clearly displays bioadhesive properties, in particular in the test using mucin to simulate the pharyngeal and oral mucosa.

The aim of the artificial saliva immersion test is to show the contact time of the product with the biological membrane when subjected to the effect of saliva. As before, the product is deposited on the surface of a biological membrane covered in mucin. After flowing over a distance of 10 cm, the plate/biological membrane/product assembly is submerged vertically in a container with 2 litres of artificial saliva. The time the product remains on the surface of the membrane is determined using a chronometer. The measured times are as follows:

| | |
|---|---|
| Distilled water | <1″ |
| Liposome preparation | <1″ |
| Bioadhesive preparation | 64″ |

Since the liposome preparation does not adhere to the biological membrane covered in mucin, the immersion time of the product in artificial saliva is actually negative (<1″). There is a complete absence of adhesion in the presence of saliva. Conversely, in the case of the bioadhesive preparation, it displays excellent bioadhesive properties.

By modifying the flow mode of the product on the plate, it was impossible to determine an immersion time for the liposome preparation in saliva, given that the product is immediately dispersed in the artificial saliva. Conversely, by tilting the plate by 45°, the bioadhesive preparation displays adhesive properties for more than 48 hours.

A composition was examined under experimental conditions to verify the synergy existing between the lubricating substance and the bioadhesive substance on its bioadhesive properties. The study performed consists of measuring the previously defined rate of flow using different concentrations of a mixture of glycerol and 3% water (S2) as a lubricating substance for a given concentration of carrageenans (S1) as a bioadhesive substance.

This test was conducted on a 20 cm² stainless-steel plate covered in a cellulose membrane impregnated with a 5% mucin solution.

For this study, 0.5 ml was deposited 0.5 cm from the top edge of the plate, the deposit being made in horizontal position. The chronometer starting line is located 5 cm from the top edge of the plate when the liquid crosses it after tilting the plate to 45°. In the same way, the chronometer stopping line is located 5 cm from the bottom edge of the plate when the liquid crosses it. The results are as follows:

| | Flow time | Flow time (in min)/ |
|---|---|---|
| | (in min) | S2 concentration ratio |
| 2% carrageenan solution (S1) | 0.83 | / |
| 7.5% S1 + S2 (mixture | 14 | 1.9 |
| of glycerol and 3% water) | | |
| 10% S1 + S2 | 18.6 | 1.9 |
| 16% S1 + S2 | 70 | 4.4 |

The ratio of flow time to the concentration of S2 is constant (1.9) for S2 concentrations of up to 10%, and increases considerably for concentrations of around 16%. This clearly shows that for a given solution containing 2% of carrageenans, the addition of glycerol exponentially increases the flow time, which is a measure of the bioadhesiveness of the final mixture.

This table clearly shows the boosting effect of glycerol on the bioadhesiveness of carrageenans.

The technology described above is representative. It is understood that those skilled in the art are capable of creating different variations of the compositions without departing from the scope of the appended claims.

1-20. (canceled)

21. An anti-snoring composition comprising at least one lubricating substance and at least one bioadhesive substance which causes the lubricating substance to adhere to the mucocilliary cells located at pharynx level.

22. The composition according to claim 21, wherein the bioadhesive substance is at least one selected from the group consisting of polysaccharides, cellulose derivatives, acrylic derivatives and protein derivatives.

23. The composition according to claim 22, wherein the bioadhesive substance is a polysaccharide belonging to the carrageenan family.

24. The composition according to claim 21, containing about 0.5 to about 20% of bioadhesive substance.

25. The composition according to claim 24, containing about 1 to about 5% of bioadhesive substance.

26. The composition according to claim 24, containing about 5 to about 20% of bioadhesive substance.

27. The composition according to claim 21, wherein the lubricating substance is a surface-active agent with a polar part and a hydrophobic part.

28. The composition according to claim 27, wherein the polar part of the surface-active agent is at least one selected from the group consisting of glucose, sucrose, lactose, glycerol, xylose, peptides, amino acids and nucleotides.

29. The composition according to claim 27, wherein the hydrophobic part is at least one selected from the group consisting of fatty acids, fatty alcohol, fatty amine, esters, glycerides and phospholipids.

30. The composition according to claim 29, wherein the hydrophobic part comprises phospholipids.

31. The composition according to claim 21, containing about 0.5 to about 10% of lubricating substance.

32. The composition according to claim 31, containing about 2 to about 20% of lubricating substance.

33. The composition according to claim 21, further comprising one or more anti-inflammatory and/or tonic agent(s) for the mucosa and tissue of the pharynx.

34. The composition according to claim 33, wherein the anti-inflammatory agent is a Filipendula ulmaria extract.

35. The composition according to claim 34, wherein the extract is an extract of Filipendula ulmaria flowering tops.

36. The composition according to claim 33, containing about 0.5 to about 8% of anti-inflammatory agent.

37. The composition according to claim 33, wherein the tonic agent for the mucosa and tissue of the pharynx is rosehip.

38. The composition according to claim 33, containing between about 0.5 and about 8% of tonic agent.

39. The composition according to claim 21, in the form of a foam.

40. The composition according to claim 21, containing:
about 1 to about 5% of carrageenans,
about 1 to about 10% of phosphatidylcholine,
about 1 to about 5% of Filipendula ulmaria,
about 1 to about 5% of rosehip.
